(19) United States
(12) Patent Application Publication
(10) Pub. No.: US 2006/0149594 A1
(43) Pub. Date: Jul. 6, 2006

(54) Health care facility admission control system

(75) Inventors: Donavon Hilligoss, Galesburg, IL (US); Richard Dechow, Galesburg, IL (US); David Dechow, Abington, IL (US); Jerry Hawley, Newport Beach, CA (US); Ron Debus, Kailua, HI (US)

Correspondence Address: HUSCH & EPPENBERGER, LLC, 190 CARONDELET PLAZA, SUITE 600, ST. LOUIS, MO, 63105-3441, US

(73) Assignee: Healthcard Network, Galesburg, IL (US)

(21) Appl. No.: 11/027,865

(22) Filed: Dec. 30, 2004

(51) Int. Cl. G06Q 10/00; A61B 5/00

(52) U.S. Cl. 705/2; 600/300

(57) ABSTRACT

A system and method for admitting a patient to a health care facility is disclosed. The method includes the steps of: providing a computer having a memory and a cache storage area; connecting a proximity sensor to said computer; beginning a computer session for the patient; inputting personal data relating to the patient; tripping said proximity sensor; and sending a signal to said computer, whereby upon receipt of said signal said personal data is saved to said memory, said computer session is automatically terminated and said cache storage area is automatically cleared.

### CROSS-REFERENCE TO RELATED APPLICATIONS

None.

### STATEMENT REGARDING FEDERALLY SPONSORED RESEARCH OR DEVELOPMENT

Not Applicable.

### APPENDIX

Not Applicable.

### BACKGROUND OF THE INVENTION

1. Field of the Invention

This invention relates generally to automated health care management and, more specifically to a system and method for admitting a patient to a health care facility with proper personal information being recorded.

2. Related Art

Management of patient identity and financial data in the fields of medical, dental, ophthalmological, podiatric, chiropractic, pharmacological and other health care areas, has become a complex, expensive and time-consuming aspect in the provision of health care services. Hospitals and Health Care Professionals must divert valuable time, energy and resources to address paperwork and the complicated field of data management. Accordingly, health care providers are unable to direct as much time to the provision of health services as they otherwise would. The cost of providing patient care has increased while reimbursement has decreased. Insurance companies have gained an ever increasing presence in every field of health care as well as service industries, providing for the vast majority of fee payments. Multipe forms, requests and releases must be accurately filled out for each individual patient in order for the health service provider to be reimbursed for the care rendered.

When a patient sees a new doctor or seeks treatment in a clinic or hospital for the first time, and generally every time thereafter, it typically takes the service or care provider, or their respective staff, between fifteen (15) minutes to one (1) hour to fill out all the forms, questionnaires, check the applicable sources and facts, check the information's accuracy and the completeness of all the above mentioned details. Additionally, for many reasons, it is often necessary to check with the insurance company, previous service providers, clinics and hospitals to insure the completeness, accuracy and veracity of the information provided. In many instances, information and verification of it must be obtained without the patient's/insured's help, and is therefore difficult to obtain quickly. Generally, the only readily verifiable identification that a patient carries is a driver's license. The large number of managed care companies with varying rules and programs have confused matters further.

Identification issues aside, managed care, private insurance, business insurance plans and government sponsored health care generally account for payment of the vast majority of patient fees. Billing procedures are generally computer managed in virtually all doctor's practices, laboratories, emergency rooms, hospitals and clinics. Electronically filed claims expedite the processing and payment of many claims submitted. Major insurance carriers, as well as state health care programs and Medicare, encourage electronically filed claims. Medicare and some insurers will only accept electronic claim filing. Medicare is presently accepted by 90% of physicians and essentially all hospitals, clinics and labs. Additionally, electronically filed claims vastly reduce the amount of unnecessary paper that would otherwise be required. Furthermore, due to the progressive aging of our society such electronic claims will rise out of necessity.

A problem with the filing, processing and satisfaction of any electronically filed claim is that all the information must be absolutely correct and the format must be in full compliance with the requirements of the insurer. Such errors may result in the insurance carrier's outright refusal or significant delay in payment for the care or service provided. Common causes of claim refusals include inaccurate identifying information or addresses for patients, incomplete forms, incorrect identification of a primary payor, lack of a medical necessity for Medicare and incorrect procedure codes.

At present, in a vast majority of the offices, patients complete questions on handwritten forms. A receptionist, who is usually not trained in data entry, must enter patient and insurance information into a computer while concurrently accomplishing and performing many other tasks. Errors in data translation and entrance occur frequently because of patient and/or provider employee error. In the event procedures (for example lab tests, biopsies, consultations or, blood specimens) are performed or ordered, a patient's information and insurance's data must be again transcribed, providing another opportunity for error.

Further errors are caused by uncoordinated patient information databases and by multiple hospital admission locations at a single hospital.

All errors and/or omissions must be corrected before the insurance claim is paid. Such corrections require meticulous and time consuming review and additional phone calls that result in further delay in claim payment —if payment is remitted at all. Additional employees are often hired in a stop gap attempt to cope with errors, call insurance companies, review the patient's files and review all the aforementioned work to check and verify it. In turn, the additional employees, paperwork and support mechanisms tend to interfere with the normal flow of patients and rendering of care. Furthermore, many people have substantial difficulty filling out the long forms whereas others simply refuse to fill out all the forms. Patients with language barriers, mental handicaps, the acutely ill and unconscious patients are unable to complete the required forms for authorization of payment and more specifically and importantly treatment. Admitting staff personnel are often overworked and undertrained.

Another complicating aspect of managed care, HMOs or PPOs, is the fact that each payment provider often has several programs with different requirements, restrictions, codes, forms and even several different billing addresses. The above-mentioned problems cause medical care providers to be reluctant to comply with any additional record keeping and reporting requirements, especially in the midst of busy patient care. The significant burdens associated with the time, cost and the amount of paperwork required for proper patient account processing cause many physicians and institutions to reject particular insurance plans and carriers altogether.

A need has arisen for a method to assure accurate and complete identification, demographic, insurance and credit information on patients, which may also include basic “medical-alert” information.

Admitting systems must also comply with the Health Information Patient Privacy Act. There is a continuing need to prevent identity theft and to protect medical information from improper disclosure.

There is a further need for streamlining re-admission procedures, for recognizing pre-authorized and previously admitted patients and automatically populating their forms.

### SUMMARY OF THE INVENTION

It is in view of the above problems that the present invention was developed. The invention is a system, data structure and method for admitting a patient to a health care facility. The system includes a computer having a memory. A monitor, an input device, an identification scanner, and a proximity sensor are all connected to the computer. The computer displays personal data questions to the patient via the monitor, and in response the patient enters personal data through the input device. The memory is adapted to store the inputted personal data of the patient. Additionally, the identification scanner, such as a biometric scanner, is used to identify the patient and match the patient with a data entry stored in the computer. In this manner, the patient can quickly and easily be registered for re-admission.

The proximity sensor signals the computer when the patient starts and stops using the computer. The proximity sensor is triggered when the patient steps away from the computer. Upon receiving the signal, the computer can carry out various functions. For example, the computer may save the personal data to the memory upon receiving the signal.

Further features and advantages of the present invention, as well as the structure and operation of various embodiments of the present invention, are described in detail below with reference to the accompanying drawings.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1 is a perspective view of a kiosk of the present invention;

FIG. 2 is a top view of a plurality of kiosks of the present invention;

FIG. 3 is a flow chart;

FIG. 4 is a first form;

FIG. 5 is a second form;

FIG. 6 is a third form;

FIG. 7 is a patient ID verification flow chart;

FIG. 8 is a preauthorization flow chart;

FIG. 9 is a re-admission flow chart;

FIG. 10 is a primary payor flow chart; and

FIG. 11 is block diagram showing an overview of the system.

### DETAILED DESCRIPTION OF THE PREFERRED EMBODIMENTS

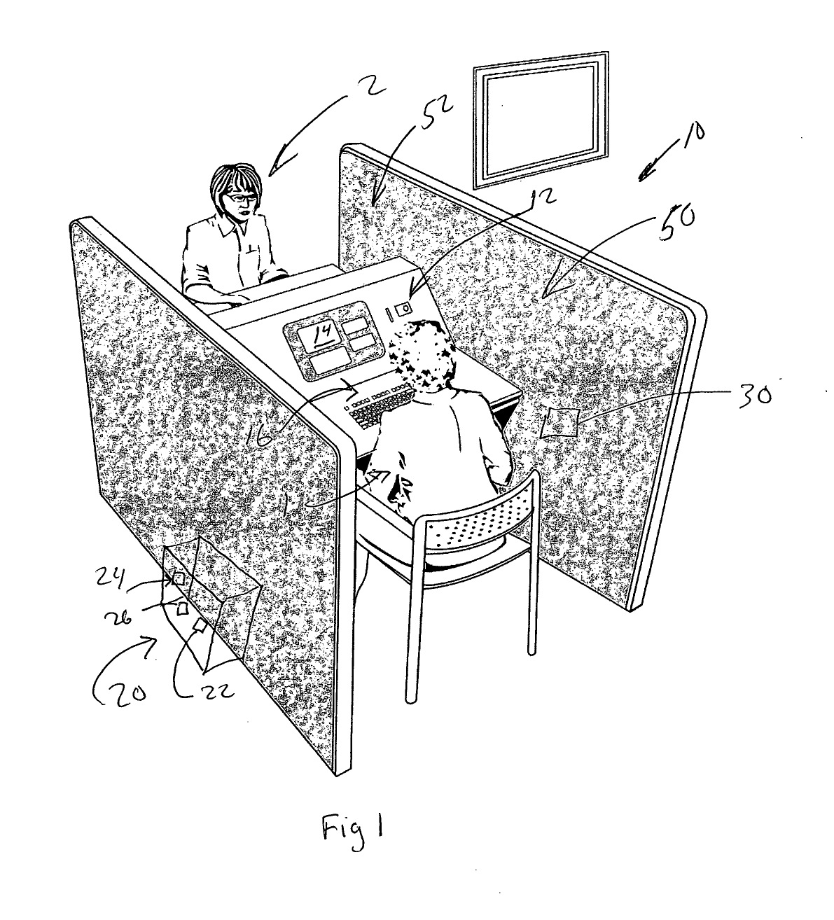

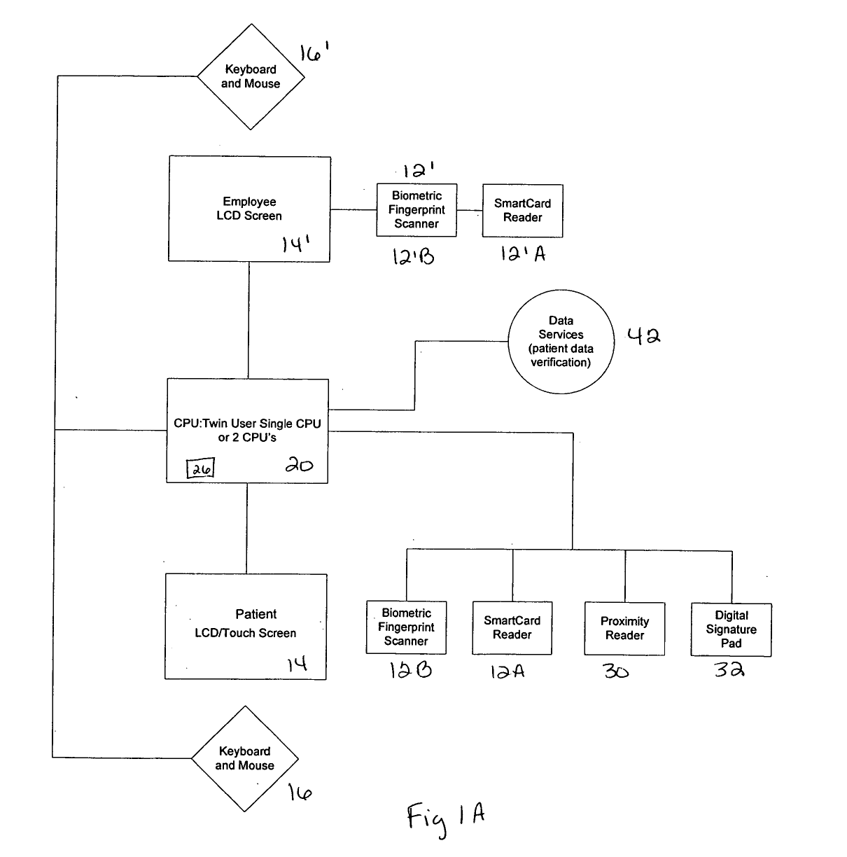

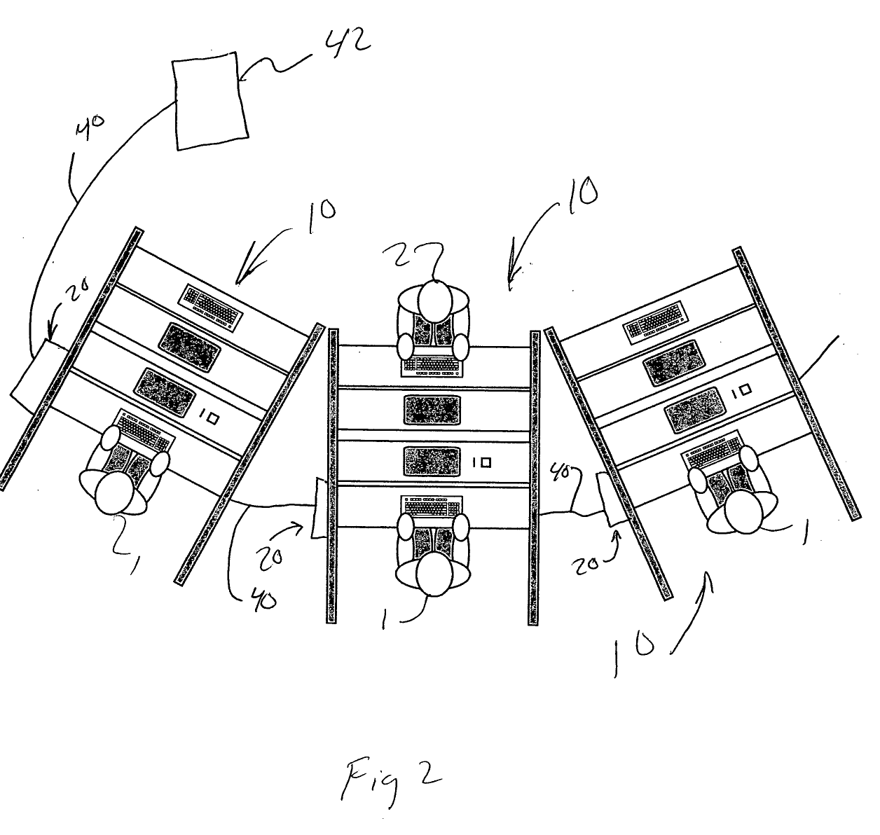

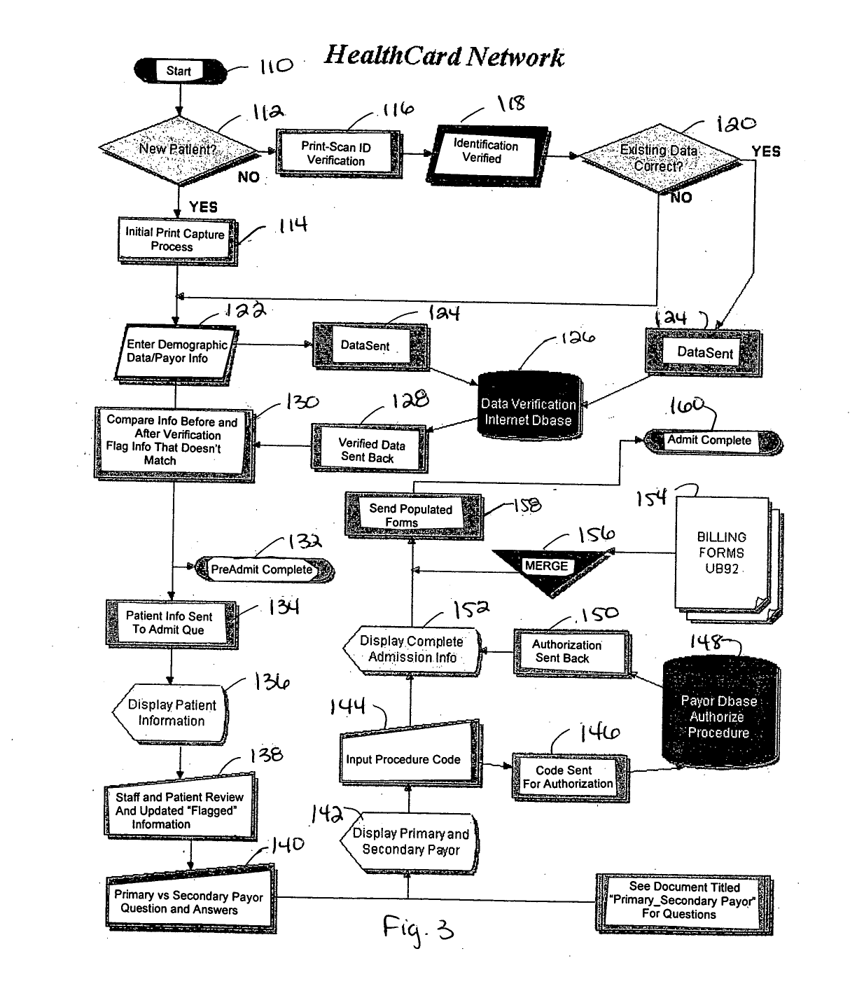

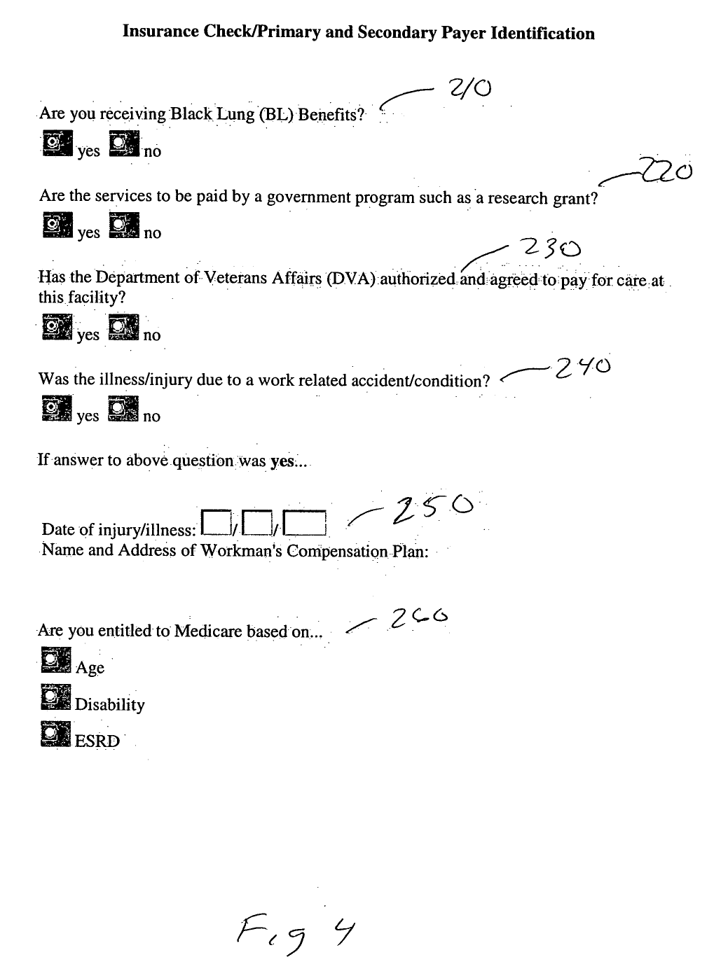

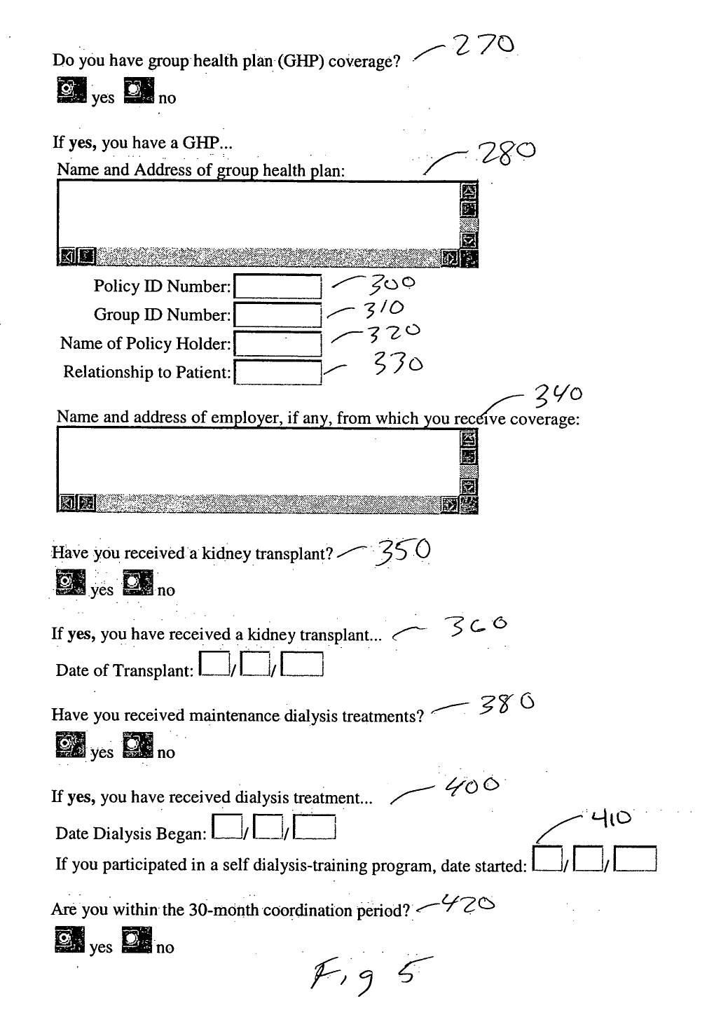

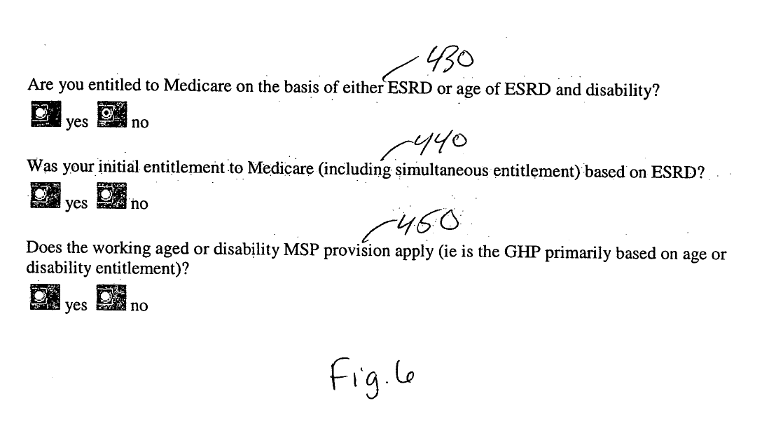

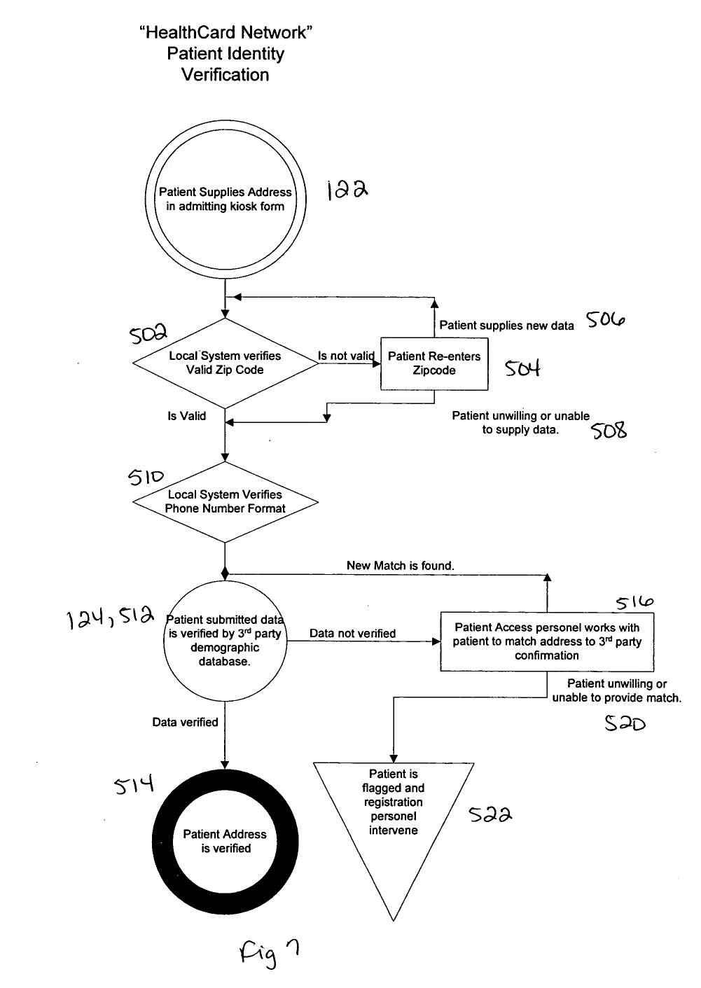

Referring to the accompanying drawings in which like reference numbers indicate like elements, FIG. 1 is a graphic depiction of a kiosk or cubicle 10 having an identification scanner 12, a monitor 14, an input device 16, and a proximity sensor 30. In the embodiment depicted in FIG. 2, there is a plurality of kiosks 10 which many patients 1 can use to start the admission process. Each kiosk is schematically depicted in FIG. 1A. A patient 1 utilizes a first side 50 of the kiosk 10 to input certain personal data or information in response to a personal data question during the admission process. An administrator 2 utilizes a second side 52 of the kiosk 10 to review the data inputted by the patient 1. In the embodiment depicted in FIG. 2, the administrator 2 may use the second side 52 to monitor more than one patient.

The second side 52 is a mirror-image of the first side 50. In other words, the second side 52 also includes an identification scanner 12′, a monitor 14′, and an input device 16′. The administrator 2 uses the monitor 14 to review the data input by the patient 1, and the identification scanner 12 may be used to verify the identity of the administrator 2 for security purposes. However, in some embodiments, the identification scanner 12 and the proximity sensor 30 may be omitted. The administrator's monitor may differ from the patient's by flagging incomplete or incorrect data fields, notifying of pre-authorization, naming benefit amounts and co-pay amounts for insurance or HMOs, displaying credit report information, notifying of Medicare as primary or secondary payor, and notifying of verification of data. Whether or not any of this information is also displayed to the patient is optional.

The identification scanner 12, the monitor 14, the input device 16, and the proximity sensor 30 are all connected to a computer 20. The computer 20 includes a memory 22 and a cache storage area 24. The personal data is stored in the memory 22. In the depicted embodiments, the computer 20 is connected to a network 40 and a server 42. As examples, the computer network 40 may be a local area network or a wide area network, such as the Internet. In the depicted embodiment, the computer 20 includes a data structure 26.

The input device 16 allows the patient 1 to input information into the computer 20. As examples, the input device 16 may be a keyboard, a mouse, or a digital signature pad and stylus. In the depicted embodiments, the digital signature pad and stylus is of the type produced by Topaz Systems, having a mailing address of 650 Cochran Street, Suite 6, Simi Valley, Calif.

The monitor 14 is used by the patient 1 or the administrator 2 to view personal data questions and the personal data input by the patient 1.

The identification scanner 12 scans the identification of the patient 1. In one embodiment, the identification scanner 12 is a smart card reader 12A. The patient 1 inserts a smart card into the smart card reader, and the smart card reader retrieves personal data from the smart card. Thereafter, the smart card reader transmits the personal data to the computer 20. U.S. Pat. No. 6,112,986 issued to Berger et al. on Sep. 5, 2000, incorporated herein by reference, discloses a method and apparatus for accessing personal data of a patient stored on a credit card-like medium. The smart card and reader may be similar or identical to the device disclosed in U.S. Pat. No. 6,112,986. The card will contain a chip that may be read by the admission computer through known hardware at the kiosk. The chip is read for its patient data. The chip may also be written to in order to update its information.

In an alternative embodiment, the identification scanner 12 is a biometric scanner. As examples, the biometric scanner may be a face scanner, a finger print scanner, a hand geometry scanner, an iris scanner, a retinal scanner, or a voice scanner. In this embodiment, the biometric scanner scans the patient 1, the biometric scanner sends the results of the scan to the computer 20, and the computer 20 matches the scan results with a stored date record stored in memory 22. As an example, the finger print scanner may be the Biocert Fingerprint Hamster III, available from Artemis Solutions Group, LLC, which is doing business as Biometrics Direct, and having a place of business in Freeland, Wash. Biometrics confirm patients' identity, identify frequent improper users of emergency rooms, eliminate identity theft and speed admissions.

Patients may confirm entered data by executing a digital signature on a digital signature pad 32.

The kiosk 10 also includes a proximity sensor 30. The proximity sensor 30 is a device that signals to the computer 20 whether a patient 1 is present at the kiosk 10. As examples, the proximity sensor 30 may be a pressure sensitive mat, a laser kill switch, a photoelectric switch, an ultrasonic switch, or a fiber optic switch. As an example, the proximity sensor 30 may be the ULTRA 100 produced by Senix® Corporation, having a postal address of 52 Maple Street, Bristol, Vt. The proximity sensor 30 is triggered when the patient 1 leaves the kiosk 10. When the proximity sensor 30 is triggered, it sends a signal to the computer 20. This signal may blank the screen to protect the privacy of the patients' information. Upon receiving the signal, the computer 20 can carry out any of various functions. For example, the computer 20 may save all of the patient's personal data to memory 22. In another example, the computer 20 may erase or clear the cache storage area 24. In yet another example, the computer 20 may log out the patient 1. In other words, the computer 20 will automatically terminate the computer session. Moreover, the computer 20 may carry out a combination of functions upon receipt of the signal from the proximity sensor 30. For example, the computer 20 may both save all of the patient's personal data to the memory 22 and clear the cache storage area 24. Alternatively, the computer 20 may save the personal data to the memory 22, log out the patient 1, and erase the cache storage area 24. The various functions may be carried out by Account Management Module 26 data structure.

FIG. 3 illustrates a flow chart of the admission process using the kiosk 10. The patient 1 walks up to the kiosk 10 and starts the admission process in a first step 110. In some embodiments, the patient 1 may begin a computer session. Some embodiments may offer a choice of language for the patient to use. The patient 1 inputs their identity into the kiosk 10 using the identification scanner 12. In the depicted embodiment, the patient 1 uses the biometric scanner to provide the kiosk 10 with their fingerprint identification. However, those skilled in the art will understand that other methods of presenting identification can be used.

The computer 20 determines in the second step 112 whether or not the patient 1 is a new patient. This may be done by the patient indicating the fact, or by an automatic data base check. If the patient 1 is a new patient, then a new patient record is established in step 114. In step 114, the computer 20 records in the memory 24 the biometric scan of the patient 1. In step 116, if the patient is not a new patient then the biometric scan is verified and matched with a data record stored in the memory 24. This is accomplished by comparing the present record with the previously recorded record in a routine depicted in FIG. 7. In step 118, the identification is verified by establishing that the records match. In step 120, there is a decision whether or not the existing data associated with the record is correct. If the existing data is correct, then data is sent in step 124 to a data verification database in step 126. However, if the existing data is not correct then the process picks up at step 122. In step 122, for either an incorrect existing data or for a new patient, demographic data and payor information is entered. Once this is complete in step 124, data is sent to the data verification database in step 126.

The database verification database is comprised of third party clearinghouses. Third party clearinghouses are described in U.S. Pat. No. 5,832,447 issued to Rieker et al. on Nov. 3, 1998, herein incorporated by reference. In the depicted embodiment, the data is encrypted and sent to the data verification database via a computer network, such as the Internet. Additionally, the data is sent using a known standard for the exchange of data. As an example, the data may be sent using the Health Level Seven (HL7) messaging standards. In the depicted embodiment, the kiosk 10 utilizes HL7 Version 2.5, which is incorporated by reference herein. (This standard is also used for communication between admitting equipment and other hospital data bases and processors.) Information that may be verified by third party services includes patient identity, correct address, Medicare medical necessity, insurance benefits availability and of course, credit checks.

In step 128, verified data is sent back to the computer 20. In step 130, the information is compared before and after verification, and information that does not match is flagged. After this has been completed, preadmission is complete as is shown in step 132. In step 134, the patient's information is sent to the admit queue, and the patient's information is displayed to the administrator 2 in step 136. In step 138, the admission personnel 2 and the patient 1 review and update flagged information to correct any information that was flagged upon data verification by the third party clearing house.

In step 140, personal data questions in the form of primary and secondary payor questions are shown on the forms shown in FIGS. 4, 5 and 6.

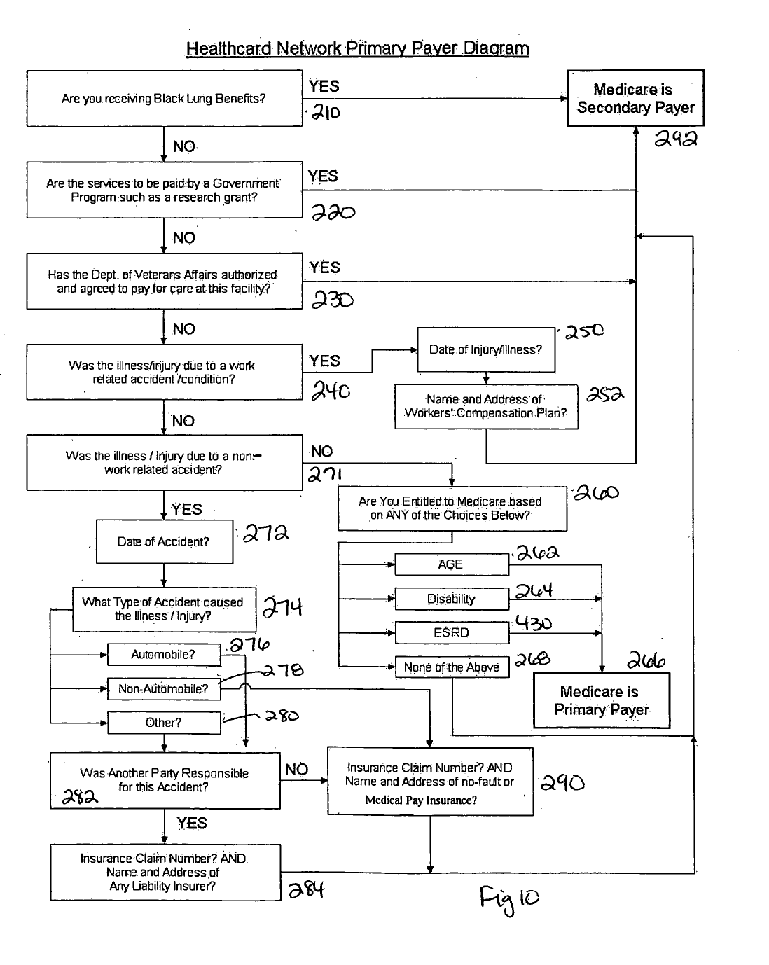

Referring now to FIG. 4 and 10, there is a first form having questions regarding primary and secondary parent information identification. Multiple forms in multiple formats may be stored, displayed for data entry, restored and re-formatted without departing from the scope of the present invention. In the first step 210, there is a question whether the patient has receiving Black Lung Benefits. In step 220, the patient 1 is asked whether the services to be paid government program, such as a research grant. In step 230, the patient is asked whether the Department of Veteran Affairs authorized and agreed to pay for care at this facility. In step 240, the patient is asked whether the illness or injury was due to a work related accident or condition. If the answer to question 240 is yes, then in step 250 the patient is asked the date of injury and illness, and the name and address of his or her Workman's Compensation Plan 252. In step 260, the patient is asked whether he or she is entitled to Medicare Benefits. The basis of that entitlement is indicated. It may be age 262, disability, 264 or ESRD (End Stage Renal Disease) 420. If the answers to any of these choices is yes, Medicare is flagged as the primary payor 266. If not, Medicare is the secondary payor 268. Optionally the patient may be asked if she is allergic to any medication.

Referring to FIG. 5 and 10, there is a second form having a series of questions relating to payer and primary and secondary payer identification. In step 270, the patient 1 is asked whether he or she has group health plan coverage. If the answer is to question 270 is yes, then in step 280, the name and address of the group health plan is requested. Additional information regarding the group health plan is requested and boxes 300, 310, 320, and 330 are provided for receipt of the additional information. For example, box 300 provides a box for a policy identification number, box 310 provides a box to put in a group identification number, box 320 provides a box for the name of the policy holder, and box 330 provides a box for relationship to patient. Box 340 provides a place to enter the name and address of the employer, if any, through which coverage may be received.

In step 350, the patient 1 is asked whether he or she has received a kidney transplant. If the answer is yes to question 350, then in step 360, the patient is asked when he or she received the transplant. In step 380, the patient 1 is asked whether he or she has received maintenance dialysis treatments. If the answer to question 380 is yes, in step 400, the patient is asked the date dialysis began and whether he or she participated in a self dialysis training program. In step 420, the patient 1 is asked whether he or she is within a 30 month coordination period.

Referring now to FIG. 6 and 10, there is a third form in which the patient is asked in step 430 whether the patient 1 is entitled to medicare on a basis of either ESRD (End Stage Renal Disease) age or disability. In step 440, the patient is asked whether his or her initial entitlement to medicare was based on ESRD. In step 450, the patient is asked whether the working aged or disability MSP (Medicare Secondary Payor) provisions apply.

Referring to FIG. 10, the patient is further prompted to answer whether the medical condition was caused by a non-work related accident 271. If the answer is no, the patient is directed to the medicare entitlement series of questions 260, outlined above. If the medical condition was caused by a non-work related accident, the patient is prompted to provide the date of the accident 272, briefly describe the accident 274 by characterizing it as an automobile accident 276 or not 278. Thereafter, if it was an automobile accident 276 or not 280 the patient is asked if another party was responsible for the accident 282. If the answer is yes, that another party was responsible for the accident, the patient is asked for the insurance claim number and other identifying information for the responsible party 284. This same series of questions is prompted when another party is responsible for an automobile accident. If it is a non-automobile accident 278, (or if the automobile accident is covered by a no fault insurance policy or mandatory no fault insurance laws), the claim number and other identifying information for no fault coverage or medical payments coverage is prompted 290. In the event any of these decision trees uncover a primary payor, medicare information is stored, but identified as being in a secondary payor status 292.

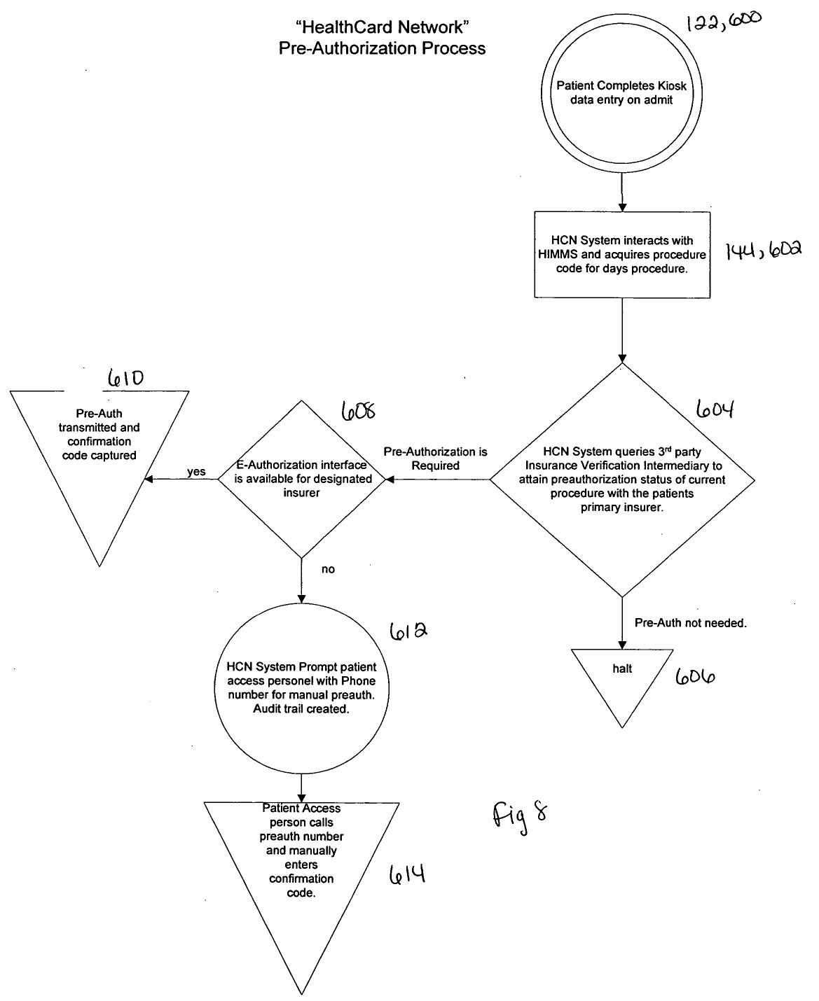

Referring once again to FIG. 3, the primary and secondary payor questions are answered in step 140, and the answers are displayed in step 142. In step 144, the administrator 2 or a physician inputs a procedure code in step 144. The procedure code relates to the procedure for which the patient 1 is being admitted. In step 146, the procedure code is sent to the payor's database for authorization. In step 148, the payor's database is checked for authorization of the procedure. The pre-authorization routine is detailed in FIG. 8, below. In step 150, authorization is sent back to the computer 20. In step 152, the complete admission information is displayed to the administrator 2. Billing forms 154 are generated. In step 156, the billing forms are merged with the admission information from step 152. In step 158, the billing forms are populated for completed forms. Thereafter, admission is complete with the completed forms in step 160.

FIG. 7 depicts the patient identity verification routine. As indicated previously, the patient supplies the address and admitting information at the kiosk form 122. Thereafter, the local system verifies a valid zip code 502. If the local zip code provider is not valid, the patient is prompted 504 to reenter the zip code to reenter the zip code, which is again verified when given 506. If the patient is unwilling or unable to supply the zip code, or if the zip code is valid at step 502, the local system next verifies the phone number at 510. With these local verifications, the patient submitted data package is forwarded at step 124, 512 to the data verification data base maintained by third party at step 512. If the proper verification is returned by the third party through the computer network, the patient is admitted 514. If the data is not verified, the healthcare administrator is notified by display to assist the patient in entering and prove data at step 516. If the patient and administrator are able to complete the data entry, the match is checked again at step 518. If the patient is unwilling or unable to provide the further information 520, the automatic verification is failed, and human registration personnel are notified to intervene 522.

The preauthorization subroutine is depicted in FIG. 8. Again, the process begins with the patient entering his identification data at the kiosk in step 122, 600. Again as previously described, the administrative personnel assist in entering procedure code 144, 602. This data packet is forwarded to the third party insurance verification intermediary to verify that the procedure is authorized under the presenting patient's coverage at step 604. In some cases, procedures do not require preauthorization, in which case the routine is halted 606. When preauthorization is required, e-mail or other electronic computer network authorization is sometimes provided by the insurer or a third party contractor of the insured. Whether or not it is available is determined at step 608. If it is, a preauthorization code is requested and returned at steps 610; If electronic authorization is not available, that fact is displayed to the hospital administrator personnel who may then use the telephone to attempt to obtain preauthorization at step 612. In either case, an authorization trail will be created. In the event that the phone call is made at step 612, the administrative assistant will enter the fact that the call was made whether authorization was received or not. If authorization is received by telephone, the healthcare personnel enters it into the record at step 614 and the routine is completed.

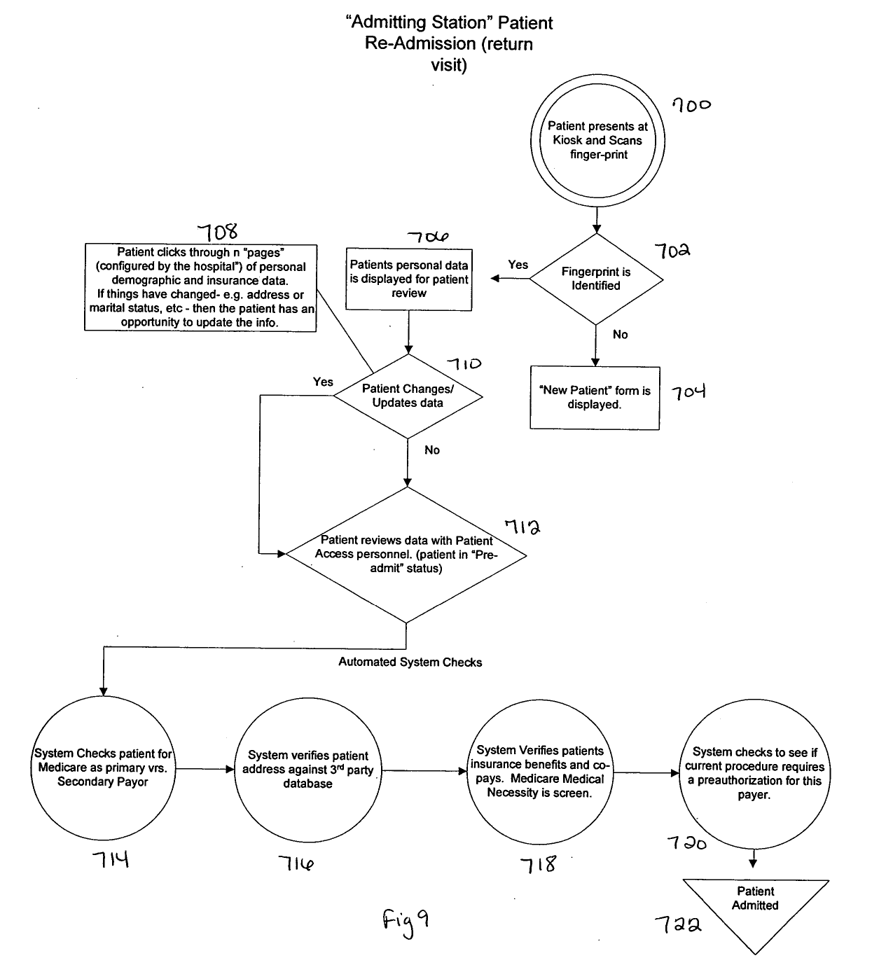

FIG. 9 depicts the routine for a patient's readmission upon a return visit. The patient presents at the kiosk and scans a biometric, for example a finger print. The biometric device either identifies the patient or not. In the event the biometric device does not identify the presenting patient, a new patient form is displayed and the patient will proceed to enter data into it. If biometric identification is confirmed, the patient's data is displayed 706 from the memory recording all data as last entered. The patient updates the data if necessary as prompted through any appropriate format 708. The patient will either update some information or not 710. Optionally incorrect information or blanks may be flagged for the admission staff's attention. In either case, the hospital admitting administrator has the most current patient data displayed 712. Initiation through the administrator, the patient or through automatic systems are all within the scope of the present invention. The hereinbefore described automated system checks are again executed. The primary versus secondary payor routine is executed 714. The third party database patient identification and address routine is executed 716. The system verifies insurance benefits and updates and displays co-pay data 718. The preauthorization routine is run again 720. Upon completion of all these routines, the patient is admitted 722.

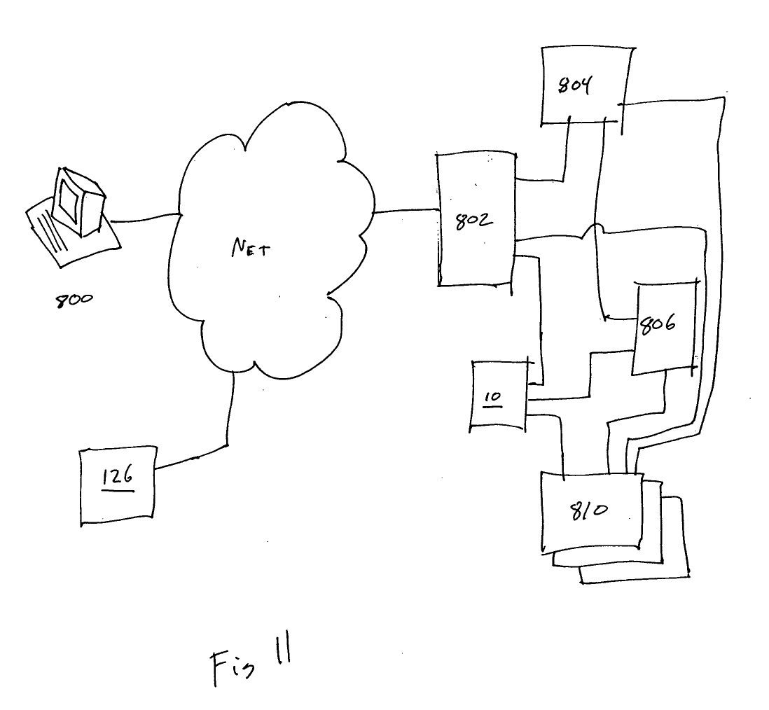

As shown in FIG. 11, the overall system is comprised of a central processor or series of linked processors 810 at the hospital. Processor 810 is linked with kiosk 10 and also linked with an interface 802 giving it access to the Internet. The central processor(s) is also linked to the permanent memory 806 for long term storage of patient data and finally linked to a separate memory space 804 which may be used for temporary storage of data. Temporary storage may include data as it is being entered, data that has not been verified and subject to further investigation, and data received by the system over the Internet.

Through network interface 802, which is constructed and arranged in any of a wide variety of known fashions, that may include Ethernet connections, firewalls and the like, the hospital system has access to the Internet. Through the Internet, as described above, it may access data from third party verification data bases 126.

Also through the internet, the system may receive data directly from patients at remote terminals 800. Such information would include preauthorization data. In operation then, before going to the hospital, the patient would access the Internet at terminal 800, select the same series of screens described above that would be available at the kiosk by going to the hospital's website to access them. Thereupon the user may enter all the same data. The system will receive this data through interface 802 and store it in temporary memory 804. Thereafter, when the patient arrives at the hospital and identifies himself in the above described manner at kiosk 10, the central processor 810 may access the short term memory 804, verify its proper correspondence to the presenting patient, and thereafter store it in long term memory 806.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. For example, the computer may carry out one or a combination of functions upon receiving the signal from the proximity sensor. Thus, the breadth and scope of the present invention should not be limited by any of the above-described exemplary embodiments, but should be defined only in accordance with the following claims appended hereto and their equivalents.

What is claimed is:

1. A health care facility admission control system for admission of a patient, the system comprising:
a. at least one computer having a memory, said memory adapted to store personal data of the patient;
b. at least one monitor operatively connected to said at least one computer, said at least one monitor adapted to display a personal data question;
c. at least one input device operatively connected to said at least one computer, said at least one input device adapted to input said personal data and wherein the patient provides said personal data in response to said personal data question;
d. at least one identification scanner operatively connected to said at least one computer; and
e. at least one proximity sensor operatively connected to said at least one computer, whereby, upon triggering of said at least one proximity sensor, said at least one computer automatically saves said personal data in said memory.

2. The health care facility admission control system according to claim 1, wherein said at least one proximity sensor is selected from the group consisting of a pressure sensitive mat, a laser kill switch, a photoelectric switch, an ultrasonic switch, and a fiber optic switch.

3. The health care facility admission control system according to claim 1, wherein said at least one identification scanner is a smart card reader.

4. The health care facility admission control system according to claim 1, wherein said at least one identification scanner is a biometric scanner.

5. The health care facility admission control system according to claim 4, wherein said biometric scanner is selected from the group consisting of a face scanner, a fingerprint scanner, a hand geometry scanner, an iris scanner, a retinal scanner, and a voice scanner.

6. The health care facility admission control system according to claim 1, wherein said at least one input device is selected from the group consisting of a keyboard, a mouse, and a digital stylus and signature pad.

7. The health care facility admission control system according to claim 1, further comprising at least one other monitor.

8. The health care facility admission control system according to claim 1, wherein said at least one computer includes a data structure for beginning a computer session for the patient and said computer session is automatically terminated upon triggering of said at least one proximity sensor.

9. The health care facility admission control system according to claim 1, wherein said at least one computer includes a cache storage area and said cache storage area is automatically erased upon triggering of said at least one proximity sensor.

10. The health care facility admission control system according to claim 1, further comprising a computer network.

11. The health care facility admission control system according to claim 10, wherein said network is selected from the group consisting of a wide area network and a local area network.

12. A health care facility admission control system for admission of a patient by an administrator, the system comprising:
a. a computer having a memory, said memory adapted to receive inputted information from the patient;
b. a first monitor operatively connected to said computer, said first monitor adapted to display at least one personal data question to the patient;
c. a second monitor operatively connected to said computer, said second monitor adapted to display at least one personal data question to the administrator;
d. a first keyboard operatively connected to said computer, said first keyboard adapted to receive said inputted information from the patient and wherein the patient provides said inputted information in response to said at least one personal data question;
e. a second keyboard operatively connected to said computer, said second keyboard adapted to receive said inputted information from the administrator and wherein the administrator provides said inputted information in response to said at least one personal data question;
f. a first mouse controller operatively connected to said computer, said first mouse controller adapted to receive said inputted information from the patient and wherein the patient provides said inputted information in response to said at least one personal data question;
g. a second mouse controller operatively connected to said computer, said second mouse controller adapted to receive said inputted information from the administrator and wherein the administrator provides said inputted information in response to said at least one personal data question;
h. a finger print scanner operatively connected to said computer, said finger print scanner adapted to scan a finger print of the patient;
i. a smart card reader operatively connected to said computer, said smart card reader adapted to read a smart card containing personal data information of the patient and transmit said personal data information to said computer;
j. a digital signature pad and stylus operatively connected to said computer, said digital signature pad and stylus adapted to receive a signature of the patient;
k. a proximity sensor operatively connected to said computer, whereby said inputted information is stored in said memory upon operation of said proximity sensor.

13. The health care facility admission control system according to claim 12, wherein said computer includes a data structure for beginning a computer session for the patient and said computer session is automatically terminated upon triggering of said proximity sensor.

14. The health care facility admission control system according to claim 12, wherein said computer includes a cache storage area and said cache storage area is automatically erased upon triggering of said proximity sensor.

15. A health care facility admission control system comprising:
a. a first kiosk, said first kiosk comprising:
i. at least one computer having a memory, said memory adapted to store personal data of the patient;
ii. at least one monitor operatively connected to said at least one computer, said at least one monitor adapted to display a personal data question;
iii. at least one input device operatively connected to said at least one computer, said at least one input device adapted to input said personal data and wherein the patient provides said personal data in response to said personal data question;
iv. at least one identification scanner operatively connected to said at least one computer;
v. at least one proximity sensor operatively connected to said at least one computer, whereby, upon triggering of said at least one proximity sensor, said at least one computer automatically saves said personal data in said memory; and
b. at least one other kiosk, said at least one other kiosk comprising at least one other computer;
c. a network;
d. a server connected to said at least one computer and to said at least one other computer via said network.

16. The health care facility admission control system according to claim 15, wherein said at least one proximity sensor is selected from the group consisting of a pressure sensitive mat, a laser kill switch, a photoelectric switch, an ultrasonic switch, and a fiber optic switch.

17. The health care facility admission control system according to claim 15, wherein said at least one identification scanner is a smart card reader.

18. The health care facility admission control system according to claim 15, wherein said at least one input device is selected from the group consisting of a keyboard, a mouse, and a digital stylus and signature pad.

19. The health care facility admission control system according to claim 15, further comprising at least one other monitor.

20. The health care facility admission control system according to claim 15, wherein said network is selected from the group consisting of a wide area network and a local area network.

21. The health care facility admission control system according to claim 15, wherein said at least one identification scanner is a biometric scanner.

22. The health care facility admission control system according to claim 21, wherein said biometric scanner is selected from the group consisting of a face scanner, a fingerprint scanner, a hand geometry scanner, an iris scanner, a retinal scanner, and a voice scanner.

23. A method of admitting a patient to a health care facility, the method comprising the steps of:
a. providing a computer having a memory;
b. connecting a proximity sensor to said computer;
c. inputting personal data relating to said patient into said computer;
d. tripping said proximity sensor; and
e. sending a signal to said computer, whereby upon receipt of said signal said personal data is automatically saved to said memory.

24. The method according to claim 23, wherein said computer includes a cache storage area and said step of sending a signal includes the step of clearing said cache storage area.

25. The method according to claim 23, further comprising the step of: beginning a computer session for the patient.

26. The method according to claim 25, wherein said step of sending a signal includes the step of automatically terminating said computer session.

27. A method of admitting a patient to a health care facility, the method comprising the steps of:
a. providing a computer having a memory and a cache storage area;
b. connecting a proximity sensor to said computer;
c. beginning a computer session for the patient;
d. inputting personal data relating to the patient;
e. tripping said proximity sensor; and
f. sending a signal to said computer, whereby upon receipt of said signal said personal data is saved to said memory, said computer session is automatically terminated and said cache storage area is automatically cleared.

28. The method according to claim 27, further comprising the step of: verifying said inputted personal data.

29. The method according to claim 27, further comprising the step of: creating a new data record for a new patient.

30. The method according to claim 27, further comprising the step of: obtaining payor authorization for a procedure.

31. The method according to claim 27, further comprising the step of: populating admission forms utilizing said inputted personal data.

32. The method according to claim 27, further comprising the step of: transmitting said inputted personal data to a payor.

33. The method according to claim 27, further comprising the step of: encrypting said inputted personal data.

34. A method of admitting a patient to a health care facility, the method comprising the steps of:
a. providing a computer having a memory and a cache storage area;
b. connecting a proximity sensor to said computer;
c. logging a patient into said computer;
d. inputting personal data relating to said patient;
e. tripping said proximity sensor;
f. sending a signal to said computer;
g. saving said personal data to said memory;
h. logging said patient out of said computer; and
i. erasing said cache storage area.
